(19) United States
(12) Patent Application Publication
(10) Pub. No.: US 2011/0257703 A1
(43) Pub. Date: Oct. 20, 2011

(54) Inductive Link with Ferrite Sheets

(75) Inventors: Martin J. Kerber, Innsbruck (AT); Dominik Hammerer, Innsbruck (AT); Matthias Zangerl, Oberhofen (AT)

(73) Assignee: MED-EL ELEKTROMEDIZINISCHE GERAETE GMBH, Innsbruck (AT)

(21) Appl. No.: 13/087,613

(22) Filed: Apr. 15, 2011

Related U.S. Application Data
(60) Provisional application No. 61/324,463, filed on Apr. 15, 2010.

(51) Int. Cl. A61F 11/04

(52) U.S. Cl. 607/57

(57) ABSTRACT

An arrangement is described for a partially implantable medical system. A communications coil is adapted for placement parallel to a corresponding partner coil for communication of an implant communications signal having an associated magnetic field component. An implant electronics module is adjacent to the communications coil and electrically connected with it for coupling of the communications signal. And a planar coil shield lens is between the communications coil and the electronics module to promote coupling of the communications signal between the coils and to shield the electronics module from interaction with the magnetic field component.

This application claims priority from U.S. Provisional Patent Application 61/324,463, filed Apr. 15, 2010, which is incorporated herein by reference.

### FIELD OF THE INVENTION

The present invention relates to implantable medical devices such as cochlear implant systems, and specifically to energy transfer mechanisms in such devices.

### BACKGROUND ART

A normal ear transmits sounds as shown in FIG. 1 through the outer ear 101 to the eardrum 102, which moves the bones of the middle ear 103, which in turn excites the cochlea 104. In response to received sounds transmitted by the middle ear 103, the fluid filled cochlea 104 functions as a transducer to transmit waves to generate electric pulses that are transmitted to the cochlear nerve 113, and ultimately to the brain.

Some persons have partial or full loss of normal sensorineural hearing. Cochlear implant systems have been developed to overcome this by directly stimulating the user's cochlea 104. A typical system may include an external microphone that provides an audio signal input to an external signal processing stage 111 where various signal processing schemes can be implemented. The processed signal is then converted into a digital data format, such as a sequence of data frames, for transmission by external transmitting coil 107 into an implanted processor 108. Besides extracting the audio information, the implanted processor 108 also performs additional signal processing such as error correction, pulse formation, etc., and produces a stimulation pattern (based on the extracted audio information) that is sent through connected wires 109 to an implanted electrode carrier 110. Typically, this electrode carrier 110 includes multiple electrodes on its surface that provide selective stimulation of the cochlea 104.

Existing cochlear implant systems need to deliver electrical power from outside the body through the skin to satisfy the power requirements of the implanted portion of the system. FIG. 1 shows a typical arrangement based on inductive coupling through the skin to transfer both the required electrical power and the processed audio information. As shown in FIG. 1, the external transmitter coil 107 (coupled to the external signal processor 111) is placed on the skin adjacent to the implanted processor 108. Often, a magnet in the external transmitter coil 107 interacts with a corresponding magnet in the implanted processor 108. This arrangement inductively couples a radio frequency (rf) implant communications signal to the implanted processor 108, which is able to extract from the signal both the audio information and a power component.

In such systems, it is desirable to optimize the coupling of the implant communications signal across the skin. Thus, it is beneficial to increase inductance and coupling between the external and internal coils. But, strong radio frequency fields can also lead to eddy currents in metallic components of the system such as the housing, battery electrodes, etc. In an effort to control these requirements and issues, system designers attempt to optimize various specific design factors such as:
coil properties (number of windings)
disruptive patterns in electrode geometry, orientation of coil relative to metallic parts
orientation of coil relative to metallic parts,
use of non-conductive (ceramic) housing components in proximity to the coil

### SUMMARY OF THE INVENTION

Embodiments of the present invention are directed to an arrangement for a partially implantable medical system. A communications coil is adapted for placement parallel to a corresponding partner coil for communication of an implant communications signal having an associated magnetic field component. An implant electronics module is adjacent to the communications coil and electrically connected with it for coupling of the communications signal. And a planar coil shield lens (e.g., made of a ferrite material) is between the communications coil and the electronics module to promote coupling of the communications signal between the coils and to shield the electronics module from interaction with the magnetic field component.

In specific embodiments, the communications coil may be an implantable receiver coil and the partner coil would be an external transmitter coil, or vice versa. The electronics module may include a module housing enclosing the electronics module and/or a battery arrangement providing electrical power, whereby the coil shield lens shields the module housing and/or battery arrangements to prevent eddy currents from arising due to the magnetic field component.

### BRIEF DESCRIPTION OF THE DRAWINGS

FIG. 1 shows a human auditory system with a cochlear implant.

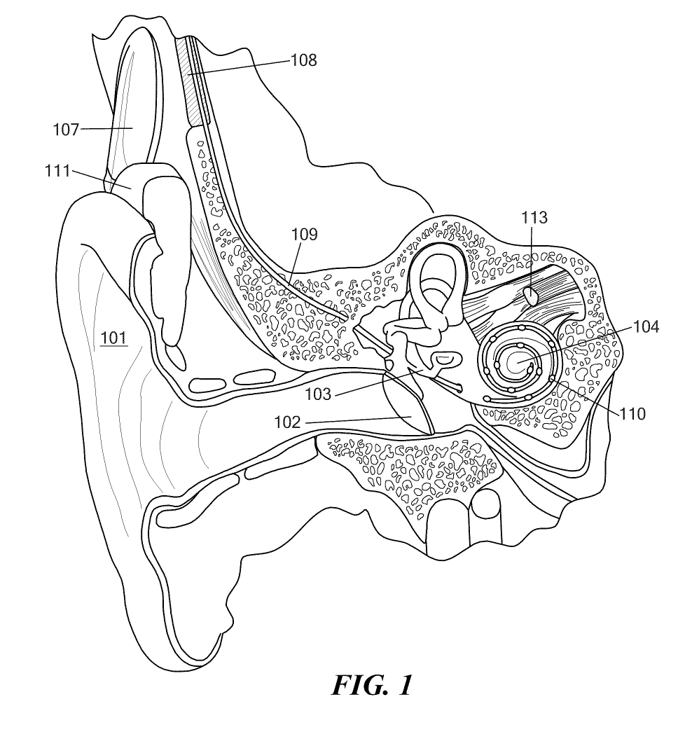

FIG. 2 A-B shows the principle of a planar coil shield lens according to an embodiment of the present invention.

FIG. 3 A-B shows photographs of a coil and a coil shield lens according an embodiment of the present invention.

FIG. 4 shows an equivalent electronic circuit arrangement of one specific embodiment of the present invention.

FIG. 5 shows coil and disk arrangement for three experimental measurements that were taken.

FIG. 6 shows a graph of data obtained for the experimental measurements shown in FIG. 5.

FIG. 7 shows various elements in a system having a planar coil shield lens according to one specific embodiment.

### DETAILED DESCRIPTION OF SPECIFIC EMBODIMENTS

Various embodiments of the present invention are directed to a magnetic shield lens arrangement for use with the communications coils in medical implant systems such as cochlear implant systems. A planar coil shield lens is positioned between one of the coils and an electronics module so as to promote coupling of a system communications signal between the coils and to shield the electronics module from interaction with the magnetic field component of the communications signal. Typically, a ferrite disk would serve as a specific example of a coil shield lens.

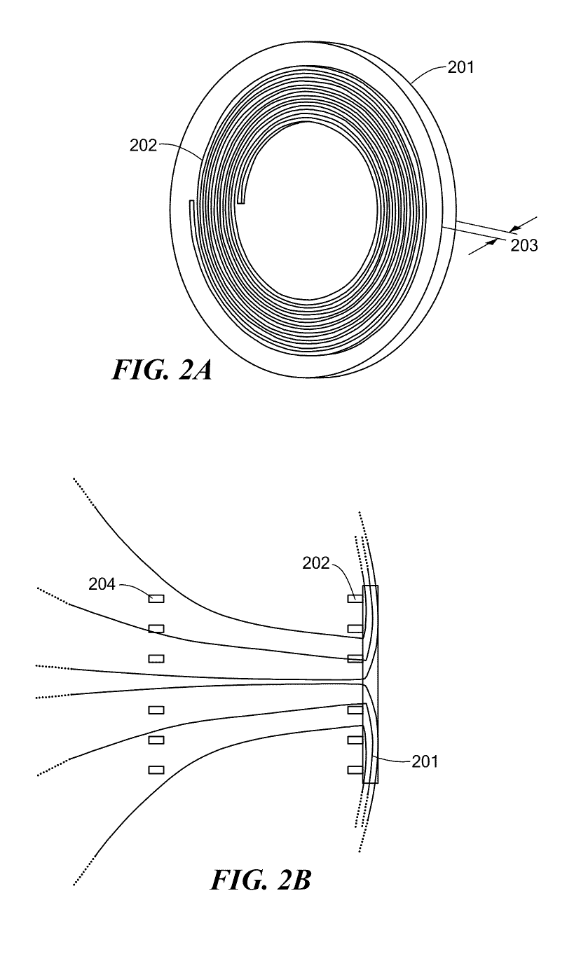

FIG. 2A shows one example of such a coil and shield lens where a generally planar communications coil 202 may be either or both of a transmitting coil or receiving coil in a medical implant system such as a cochlear implant system. The communications coil 202 is backed by a coil shield lens 201 in the specific form of a sheet of ferrite material having a lens thickness 203. FIG. 2B shows a cross-sectional view of such an arrangement together with a corresponding partner coil 204 and the lines of the magnetic field component of the communications signal between them. As seen in FIG. 2B, the field lines of the magnetic field component are focused by the coil shield lens 201 on its near side adjacent to the communications coil 202 to promote coupling of the communications signal with the communications coil 202. At the same time, the coil shield lens 201 bends the field of the magnetic field component on the far side of coil shield lens 201 to shield that area from the effects of the magnetic field component of the communications signal. For example, if an electronics module is positioned on the far side of the coil shield lens 201, then the electronics module including its housing and any components therein such as a battery power arrangement will be shielded from the potentially adverse effects of the magnetic field component such as undesirable eddy currents in the metallic components.

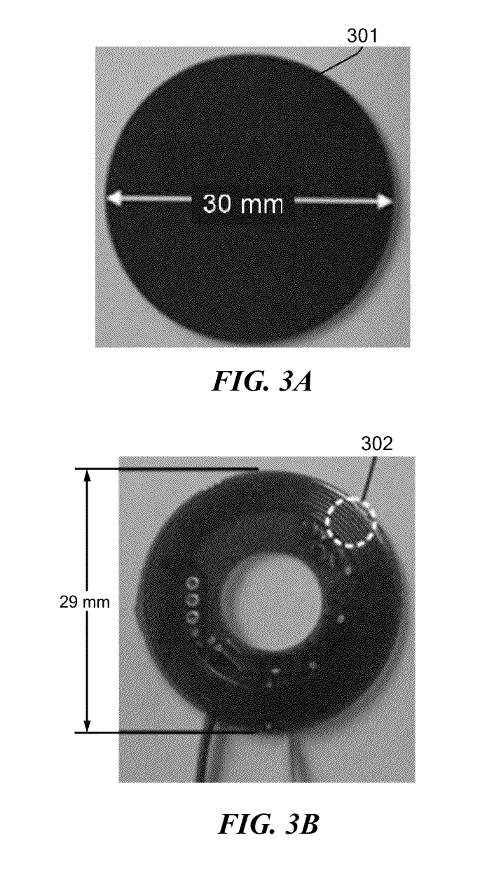

FIG. 3A shows a photograph of one specific example of a coil shield lens 301 for use in a cochlear implant system. In this case, the coil shield lens 301 is in the form of a disk of ferrite material 30 mm in diameter. FIG. 3B shows a photograph of a corresponding communications coil 302 29 mm in diameter. Once again, in specific embodiments, the communications may be either or both of a transmitting coil and/or a receiving coil.

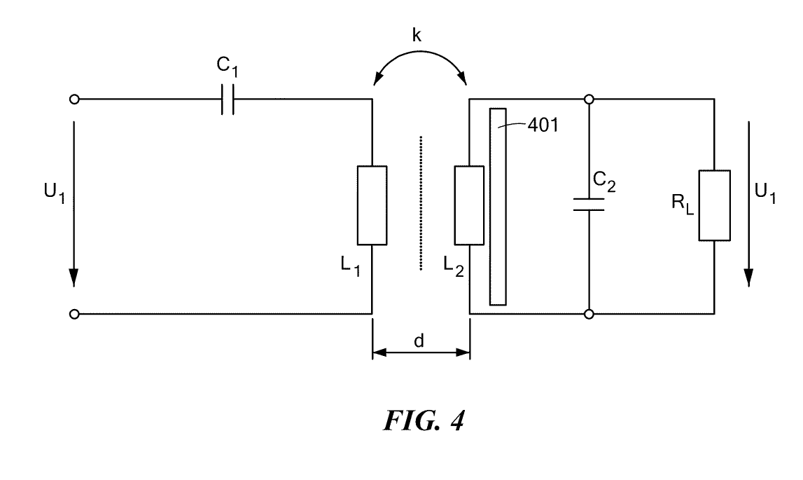

FIG. 4 shows an equivalent electronic circuit arrangement of one specific embodiment of the present invention. The depicted system is for inductive coupling of a communications signal between coils L₁ and L₂. The amount of signal coupling will be a function of the distance d between the coils and a coupling constant k. The coil shielding lens 401 acts to increase that coupling constant k to increase the coupling of the signal with respect to the physically adjacent coil L₂.

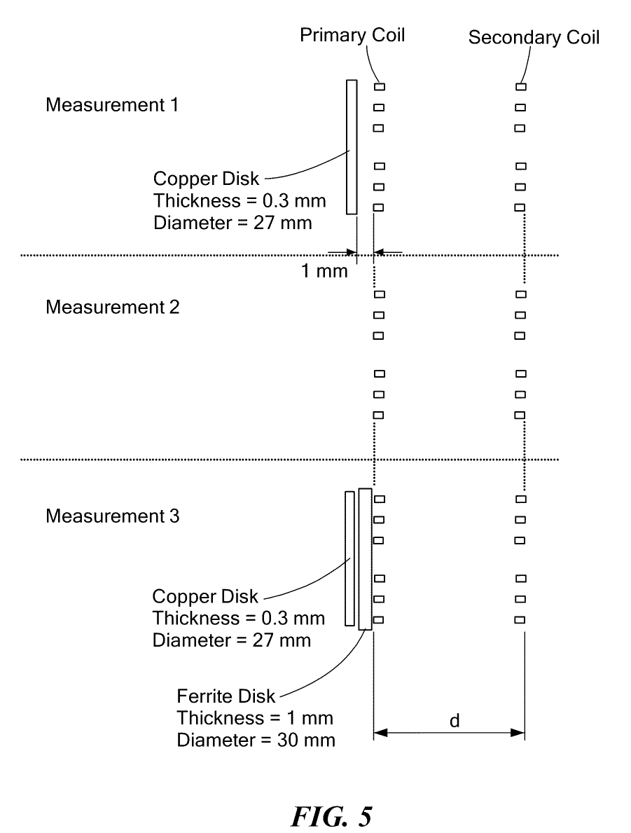

FIG. 5 shows various measurement arrangements for a series of experiments which compared the coupling effects of using a planar coil shield lens adjacent to a primary transmitting coil. In Measurement 1 of the experiments, a copper disk 3 mm thick and 27 mm in diameter was positioned in a plane 1 mm from a primary transmitting coil where it acted as a coil shield lens. Measurement 2 provided baseline data for an air coil primary without any coil shield lens. Measurement 3 used a combination of copper and ferrite disks as a coil shield lens: a copper disk as in Measurement 1 that was 0.3 mm thick, 27 mm in diameter, and 1 mm from the primary coil, and a ferrite disk 1 mm thick and 30 mm in diameter between the copper disk and the primary coil.

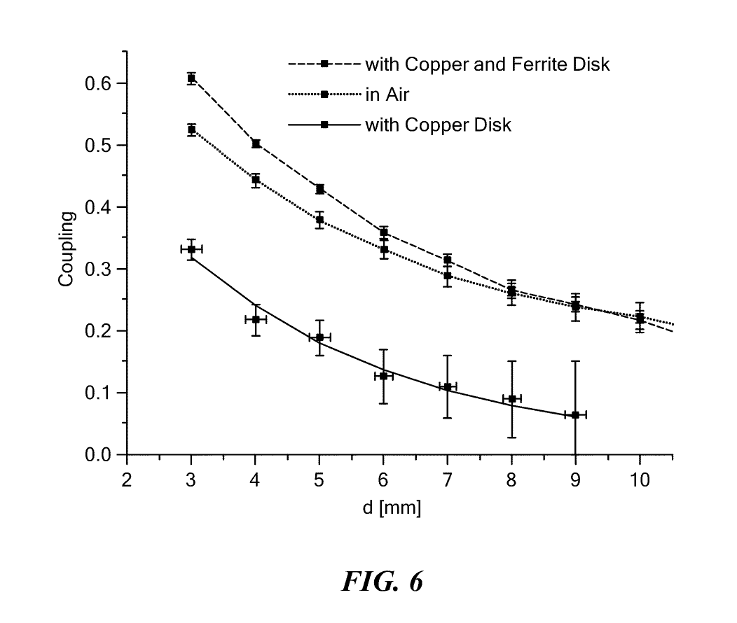

FIG. 6 shows a graph of the coupling measurements that were taken which characterized the coupling from the primary coil to a corresponding secondary coil at different distances as shown. Measurement 1 with just a copper disk had significantly lower signal coupling than for Measurement 2 without any disk. So regardless of any shielding effects on the far side of the copper disk in Measurement 1, its significantly lower coupling characteristics are undesirable. But in Measurement 3, the copper disk is supplemented with a second ferrite disk that improves the signal coupling that was observed.

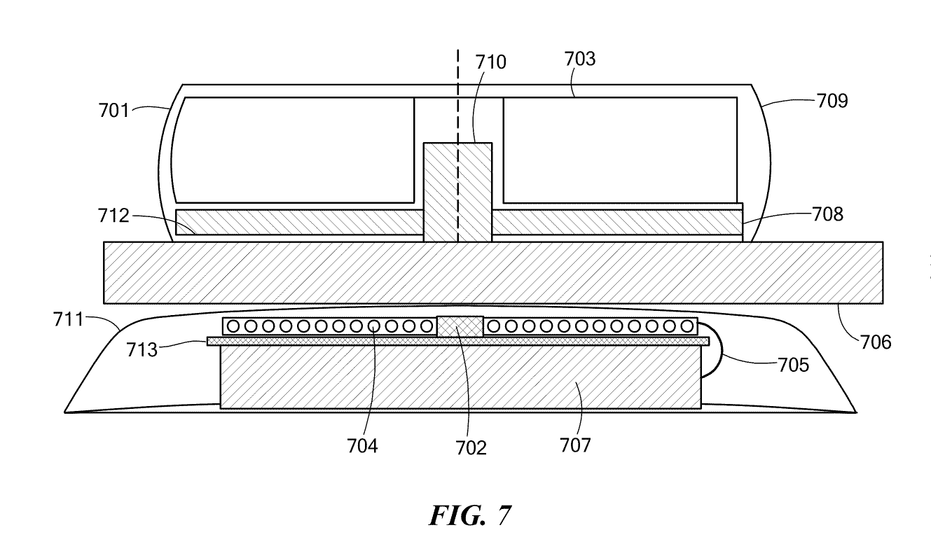

FIG. 7 shows elements of an embodiment in which an external processor housing 701 has a generally planar skin contacting surface 712. An annular housing lumen 703 contains circuitry for an external transmitter processor for developing an implant communications signal. The implant communications signal is output to a transmitter coil 708 which couples the signal across the skin 706. The housing lumen 703 also contains electrical power circuitry and a rechargeable battery arrangement which provides the power for developing and transmitting the implant communications signal. An external positioning magnet 710 is located radially inward of the housing lumen 703 and magnetically interacts with a corresponding internal positioning magnet 702 to hold the external transmitter coil 708 in a fixed position on the skin 706 over a corresponding implant receiver coil 704 in an implant housing 711. By this arrangement, the implant communications signal is coupled by the transmitter coil 708 across the skin 706 to the implant receiver coil 704. The receiver coil 704 is connected by implant wires 705 to an implant processor 707 which develops a stimulation signal for the implanted electrode array to stimulate audio nerve tissue in the cochlea.

A planar coil shield lens 713 in the form of a disk of ferrite material lies under the receiver coil 704. The coil shield lens 713 interacts with the magnetic field component of the communications signal as described above so as to increase the coupling of the communications signal to the receiver coil 704 while shielding the underlying implant processor 707 from the effects of the magnetic field component. This avoids the creation of undesirable eddy currents in the implant housing 711 and its enclosed components such as the implant processor 707 and/or any implanted battery power arrangements.

Implementing a coil shield lens as described above can significantly improve the efficiency of the RF transmission of the communications signals in medical implant systems such as cochlear implant systems. And the battery electrode plates can more readily be stacked in parallel to the coil plane without weakening of the RF transmission. In addition, since a coil shield lens prevents eddy currents from the RF signal, the materials used for the device housings can be made of metal or composite ceramic/metal structures.

Although various exemplary embodiments of the invention have been disclosed, it should be apparent to those skilled in the art that various changes and modifications can be made which will achieve some of the advantages of the invention without departing from the true scope of the invention.

What is claimed is:

1. An arrangement for a partially implantable medical system, the arrangement comprising:
a communications coil adapted for placement parallel to a corresponding partner coil for communication of an implant communications signal having an associated magnetic field component;
an implant electronics module adjacent to the communications coil and electrically connected with the communications coil for coupling of the communications signal;
a planar coil shield lens between the communications coil and the electronics module to promote coupling of the communications signal between the communications coil and the partner coil and to shield the electronics module from interaction with the magnetic field component.

2. An arrangement according to claim 1, wherein the communications coil is an implantable receiver coil and the partner coil is an external transmitter coil.

3. An arrangement according to claim 1, wherein the communications coil is an external transmitter coil and the partner coil is an implantable receiver coil.

4. An arrangement according to claim 1, wherein the electronics module includes a module housing enclosing the electronics module, whereby the coil shield lens shields the module housing to prevent eddy currents from arising due to the magnetic field component.

5. An arrangement according to claim 1, wherein the electronics module includes a battery arrangement providing electrical power, whereby the coil shield lens shields the battery arrangement to prevent eddy currents from arising due to the magnetic field component.

6. An arrangement according to claim 1, wherein the coil shield lens includes a disk of ferrite material.

7. An arrangement according to claim 1, wherein the coil shield lens includes a disk of copper material.
